(19) United States
(12) Patent Application Publication
(10) Pub. No.: US 2019/0021659 A1
(43) Pub. Date: Jan. 24, 2019

(54) Wireless Medical Evaluation Device

(71) Applicant: Renew Group Private Limited, Singapore (SG)

(72) Inventors: Ravinder Sajwan, Saratoga, CA (US); Manoj Bhargava, Farmington Hills, MI (US); Timothy Alcorn, Raleigh, NC (US)

(73) Assignee: Renew Group Private Limited, Singapore (SG)

(21) Appl. No.: 15/755,760

(22) PCT Filed: Aug. 31, 2016
(86) PCT No.: PCT/US16/49597

§ 371 Date: Feb. 27, 2018

Related U.S. Application Data
(60) Provisional application No. 62/211,915, filed on Aug. 31, 2015.
(60) Provisional application No. 62/334,675, filed on May 11, 2016.

(51) Int. Cl. A61B 5/00; A61B 5/053; A61M 37/00; A61B 5/1477; A61B 5/01

(52) U.S. Cl. CPC A61B 5/4839; A61B 5/0002; A61B 5/6833; A61B 5/684; A61B 2562/06; A61B 5/0059; A61M 37/00; A61B 5/1477; A61B 5/01; A61B 5/053

(57) ABSTRACT

A wireless medical system for remotely monitoring, detecting, and/or diagnosing a patient. The system includes a patch, a data transfer mechanism, and an assistive interpretation module. The system can also include additional modules or components without deviating from the spirit of the invention.

### BACKGROUND OF THE INVENTION

### 1. Field of the Invention

Generally, the present invention relates to the field of wireless medical devices. More specifically, the present invention relates to a wireless medical device for use in monitoring and/or diagnosing problems with a patient's health status.

### 2. Description of the Related Art

A wide variety of devices are used inside and outside hospitals for monitoring patient vital signs. One commonly used device is the Holter monitor which records heart rate, heartbeats, or rhythm continuously during a 24-hour period. The Holter monitor is a small data recorder/transmitter connected by wires to several patches containing electrodes. These patches are put on the patient's chest. The tape recorder is placed in a small protective box that fits into a case with straps so it could be easily carried on the shoulder or waist. The primary purpose of a Holter monitor is to record a patient's heart rate and rhythm during various activities over a long period. The Holter monitor is most helpful when symptoms frequently occur. It is also helpful for showing changes in heart rate or rhythm that a patient may not notice or showing increasing changes over a period of time.

Another device for monitoring patient vital signs is an event monitor. Event monitors are small, portable devices carried in a purse or attached to a belt or shoulder strap in a manner similar to that of a portable tape/digital player. When symptoms are infrequent, an event monitor may be carried for several days or a few weeks. Most monitors are designed to record the heart rate and rhythm only when a button or switch is turned on. For example, when a symptom occurs, the patient or another person could turn on the event recorder. The event recorder would then record the heart rate and rhythm. The recorded heart rate and rhythm could then be sent by telephone to a recipient in a hospital or clinic for review by a physician.

Another device, the transtelephonic monitor, is similar to an event monitor but differs in that it sends an EKG signal to a recorder by telephone. The primary purpose of both the event and transtelephonic monitors is to record the patient's heart rate and rhythm during a symptom or “event.”

All of these monitors have several significant deficiencies. First, these devices are wired and require wires running between the device and the recorder, resulting in signal artifact problems. Second, the wires could be uncomfortable to the patient. Third, water could damage the recorder, so the patient cannot swim or bathe while wearing the recorder. Fourth, these devices are large and cumbersome. And fifth, these devices merely record events; they do not provide a diagnosis of the underlying condition.

Thus, there exists a need for a compact wireless medical sensor that could a deliver performance superior to the above mentioned wired and wireless devices.

### SUMMARY OF THE INVENTION

According to the present invention there is provided a wireless medical system for monitoring, detecting, and/or diagnosing a patient remotely. The system includes a patch, a data transfer mechanism, and an assistive interpretation module. The system can also include additional modules or components without deviating from the spirit of the invention.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a top view of a wireless patch of the present invention;

FIG. 2 is a bottom view of the wireless patch of the present invention;

FIG. 3 is a cross-sectional side view of the wireless patch of the present invention;

FIG. 4 depicts a patient with the wireless patch of the present invention affixed to the patient;

FIG. 5 depicts a patient with two wireless patches of the present invention affixed to the patient;

FIG. 6 is a top view of an alternative embodiment of the wireless patch of the present invention;

FIG. 7 is a data flow chart for the system of the present invention;

FIG. 8 is a data flow chart for an alternative embodiment of the system of the present invention; and

FIG. 9 is a data flow chart for an alternative embodiment of the system of the present invention.

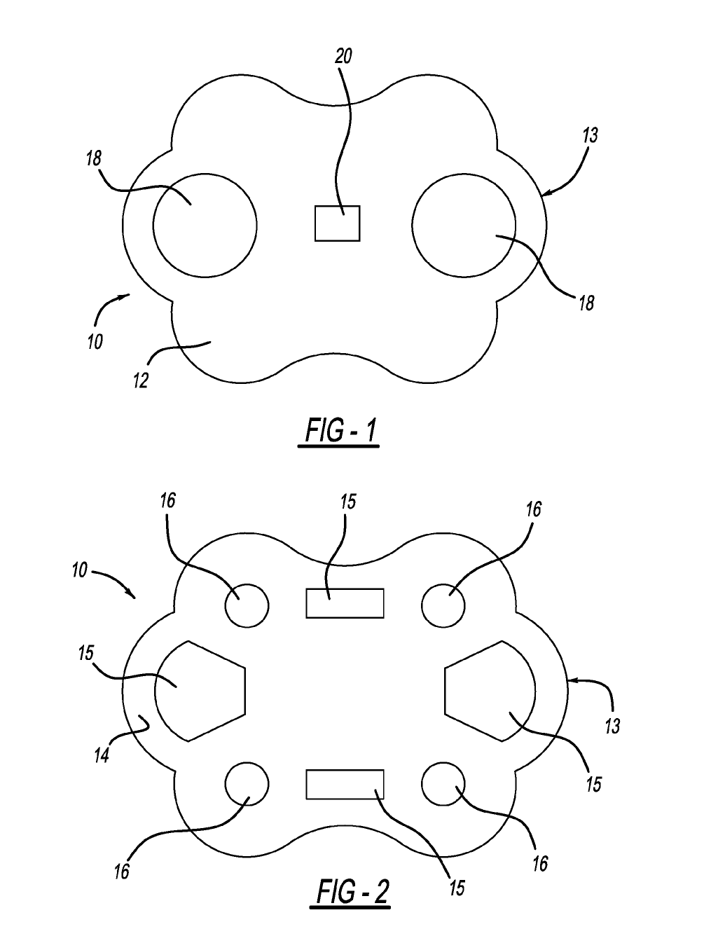

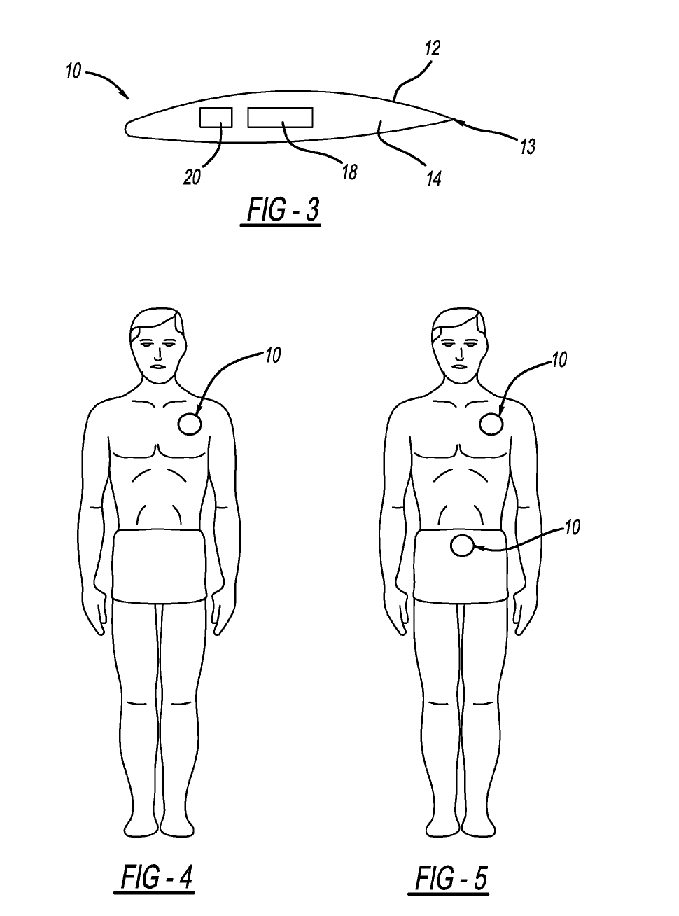

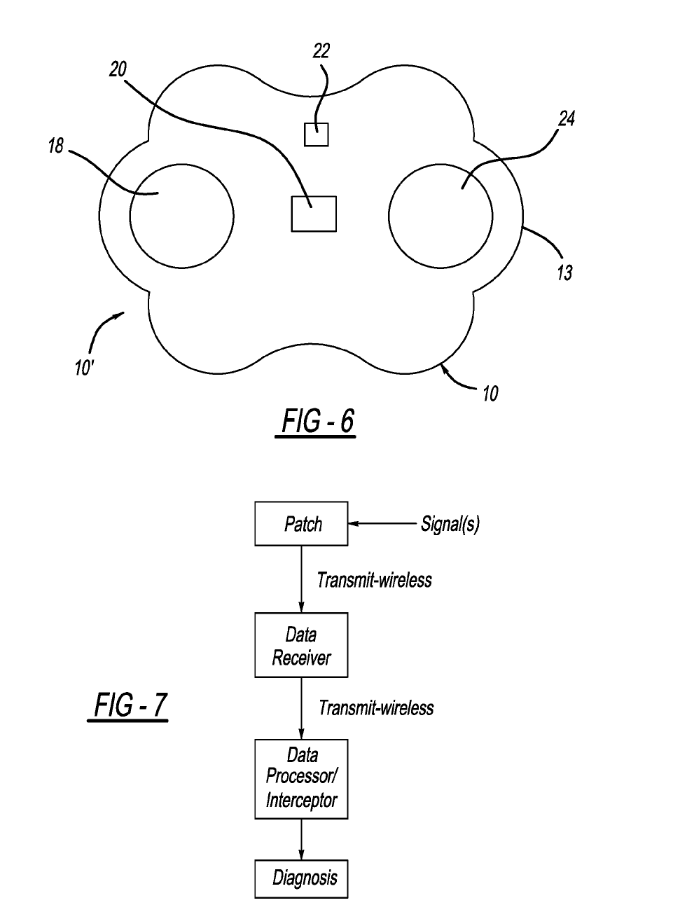

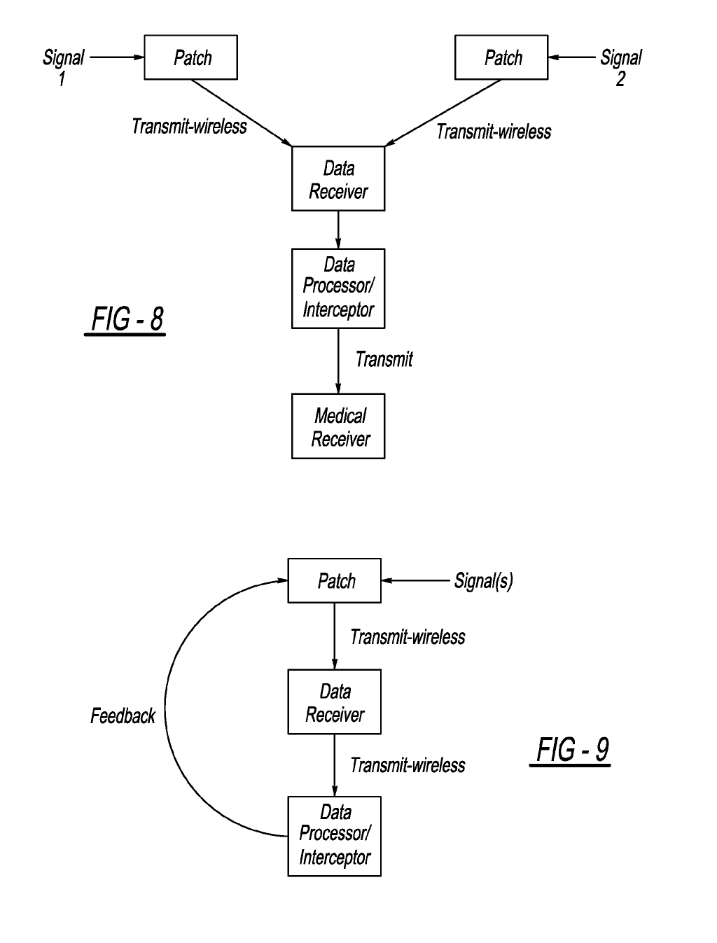

### DETAILED DESCRIPTION OF THE PREFERRED EMBODIMENTS

Generally, the present invention relates to wireless medical systems and more specifically a wireless medical telemetry system (WMTS) that enables remote patient monitoring and diagnosis. The telemetry system is used to gather and relay medically related information through interrogation of biosensors, more specifically these can be radio frequency energized biosensors (RFEB), and alternatively the medically relevant information can be transmitted using any suitable method of communicating without the use of a hard-wired electrical connection. Additionally, the system enables remote diagnosis of an underlying condition.

The present invention includes a wireless medical monitoring system, and method of using and providing the services associated with the same. The system of the present invention addresses the need for flexible, independent monitoring of a sensed signal via a self-contained and independent monitoring device that has the ability to process the sensed signal locally and then transmit the signal to one or more central collection points or base stations that integrate multiple sensor data for diagnosis. The data is then processed using an assistive interpretation module for analysis of the data; the resulting analysis is then transmitted to a doctor or other health care professional.

Alternatively, multiple patches can be used together wherein each patch monitors a separate item and then transmits the information to a single location. Each patch can include a specialized signal or other distinguishing characteristic that enables each signal to be separately analyzed; thereby ensuring that the monitored signal corresponds to the appropriate individual or location. For example, one patch can be placed on a mother, to monitor heart rate or other signals, and a second patch can be placed near a fetus to monitor the fetal heart rate. Signals from both patches then transmit the monitored signal information to a single location as disclosed herein. Alternatively, the patches can be placed at multiple locations on a single individual to monitor either different features in a single individual or to monitor the same feature at differing locations on a single individual.

A single patch can also be used in conjunction with another device, such as an echocardiogram, EKG, or other device that creates a monitorable signal or image. Similar to the use of the two patches disclosed above, the single patch with the additional device allows monitoring of multiple inputs at the same point in time. This allows a medical professional to simultaneously analyze multiple features of a patient at a set point in time, or sequentially as determined by one of skill in the art. By way of example, a patient can be monitored using the patch, which can be measuring a heartrate or other monitorable features, while an echocardiogram is simultaneously being performed. This creates a more robust understanding of the situation a patient is in at a set point in time, such that the medical professionals can better understand the totality of circumstances for a patient.

The patch, generally shown as 10 in the Figures, includes a top surface 12, is made of a durable and flexible material to allow the patch 10 to be worn for a period of time, and a bottom 14 also made of a durable and flexible material. The top 12 and bottom 14 can be made of the same or similar materials and are join with a fluid tight seam 13 to prevent materials contained within the patch from escaping as well as to prevent fluid from unintentionally entering the patch 10. The bottom 14 can contain sections of adhesive 15, or can be completely covered in adhesive, whichever is most applicable for the intended use of the patch. This can be determined by one of skill in the art. The shape of the adhesive shown in the figures is shown to provide a non-limiting example, alternative configurations are contemplated and can be used without departing from the spirit of the invention. The patch 10 also includes at least one sensor or sensor array 16. The spacing of the sensors 16 can be determined by one of skill in the art and depends upon the intended use, as disclosed herein below. The sensors 16 may be covered by the bottom 14, the adhesive 15, both the bottom 14 and adhesive 15, or not covered at all, as determined by one of skill in the art based on the intended use of the patch 10. The patch 10 also includes a battery 20 for providing energy for patch 10. The battery 20 can be any battery known to those of skill in the art to be appropriate for the intended use. The battery 20 is operably connected to an application-specific integrated circuit (ASIC) chip or chips 18. The chips are used for the wireless delivery of signals received by the sensors 16.

The devices, systems, and methods of the invention relate to the use of wireless technology. As used herein, the term “wireless” refers to any suitable method of communicating without the use of a hard-wired electrical connection. A hard-wired connection involves the direct physical connection of electrical conductors through which an electrical signal flows. The distances over which the wireless communication occurs may be short (a few meters or less) or long (kilometers or greater). The radio can be either ultra-wideband (UWB) radio or narrowband radio. Narrowband radio, as used herein, is any radio that is not ultra-wideband (UWB) radio. Some examples of the narrowband radios suitable for the present invention are: Wi-Fi standard based radio, Bluetooth standard based radio, Zigbee standard based radio, MICS standard based radio, and WMTS standard based radio. Suitable wireless radio protocols include WLAN and WPAN systems. Additionally, WMTS can include at least one battery powered module worn by the patient for collection and transmission of physiologic parameters.

The patch 10 of the present invention can utilize integrated circuits that are ASIC chips 18. An ASIC (Application Specific Integrated Circuit) is a system-on-chip semiconductor device which is designed for a specific application as opposed to a chip designed to carry out a specific function such as a RAM chip for carrying out memory functions. ASIC chips are generally constructed by connecting existing circuit blocks in new ways. The ASIC chips 18 described herein are designed for the application of monitoring of physiological signals wirelessly. In some embodiments the ASIC chips of the invention comprise two or more ASIC chips that are designed to work together. In some embodiments, the ASIC chips 18 of the present invention are made using a bulk CMOS process. In some embodiments, the ASIC chip 18 of the present invention encompasses end-to-end functionality with the following features: Analog and digital sensor electronics for multi-sensor processing; a micro-controller based design to coordinate onboard resources and perform power-control; hardwired PHY and MAC coprocessors which enable highly integrated data-paths in small silicon-area, and a CMOS radio with built-in power amplifier.

In some embodiments, the ASIC chips 18 comprise circuits for encryption and/or decryption, for advanced encryption standard (AES), cyclic redundancy check (CRC), and/or forward error correction (FEC).

In some embodiments, the ASIC chip 18 in the patch 10 is capable of receiving both analog and digital signals. In some embodiments, the ASIC chip 18 in the patch 10 supports and can receive signals from both passive and active sensing. Active signals are signals that are created by injecting an output signal and measuring a response. Active signals include radio frequency signals, electrical, optical, and acoustic signals (such as for blood-oxygen, body-impedance, ultrasound, etc.).

The patch 10 for use with the system of the present invention is a wireless patch. The patch 10 of the present invention is generally wearable. In some cases they are held onto the skin with an adhesive 15. Biocompatible adhesives are well known to those of skill in the art. In some cases, for example, where an electrical signal is being measured, e.g. for ECG or EEG, some or all of the adhesive 15 is electrically conducting, for example including silver, silver chloride, or other conductive materials known to those of skill in the art. In other embodiments, the adhesive 15 is electrically non-conductive. The patch adhesive 15 can be either wet or dry. In some embodiments, the patch can be held in place with straps or clips or may be incorporated into a piece of wearable clothing such as a hat, gloves, socks, shirt, or pants. In another embodiment, the patch is implantable. The patch 10 can be implanted subcutaneously or can be more invasive depending upon the use of the patch 10, as can be determined by one of skill in the art. When multiple patches 10 are used, for example for EEG monitoring, the patches can be kept together (in sort of a shower-cap) so that they can be applied over the head together more efficiently. In the preferred embodiment the patch 10 is disposable, but reusable patches can also be used without departing from the spirit of the invention. The patch 10 and any adhesive 15 used therewith are biocompatible and can be affixed to the skin of the patient without causing harm to the skin surface. The patch 10 includes an adhesive 15 so that the patch 10 can be affixed to the patient in an appropriate location, which location depends upon the needs of the individual patient. Alternatively, the patch 10 can be affixed adjacent the patient but within closer enough proximity to sense the necessary parameters. Examples of wireless patches are disclosed in U.S. Pat. No. 8,926,509, U.S. Pat. No. 8,688,189, U.S. Pat. No. 8,838,218, U.S. Pat. No. 7,206,603, U.S. Pat. No. 8,688,189, U.S. Pat. No. 8,718,742, U.S. Pat. No. 8,628,020, US. Pub. No. 2009/0143761, and other similar devices as are known to those of skill in the art.

The wireless patches 10 have integrated therein technology capable to monitoring select patient parameters, referred to herein as sensors 16. The patches 16 can include one or several of the following: sensor arrays, including but not limited to potentiometric, amperometric, optical sensors, biochemical, electrical, and other physical/biological characteristics associated with health or disease states, micro-electro-mechanical systems (MEMS), micro-fluidics, a delivery system, wireless communication technology, an energy source, a microprocessor and other similar systems. The sensor arrays 16 can be designed to analyze a variety of different signals. Additionally, the sensor array 16 can include multiple different detect sensors that are each capable of detecting different signals. Examples of such signals include, but are not limited to, electrical pulses, motion, such as vibrational motion, pressure, heart rate, ECG, respiration rates, temperature, blood pressure, ion and chemical concentrations, such as CO₂: SpO₂, and specific medication levels present in the body, levels of hydration, tissue impedance, accelerometer, PT-INR signals, airflow volume, hormone levels CBC, enzyme concentration, as an indicator of a disease state, salt content, blood glucose, blood oxygen content, blood pressure, acoustic monitoring of lung function, respiration rate, occlusion such as an occlusion of air flow the lung and blocked blood flow in veins or arteries, adrenal level, acetylcholine level, temperature, sodium levels, activity level for obesity and geriatric care, three axis acceleration to detect falling, and other similar detectable features.

The patches 10 may be placed on any suitable part of the body depending on the physiological signal and the condition to be measured. For ECG monitoring, for example, in some embodiments, a single patch 10 can be used, the patch 10 is placed on the upper-chest area. For EEG monitoring, e.g. for monitoring sleep Apnea, multiple patches are placed on the head. For temperature monitoring, the sensors can either be incorporated into a single patch or can be part of a secondary patch that monitors the temperature at a different location and transmits the temperature data to the main patch (examples of such as patch is disclosed in U.S. Pat. No. 8,930,147, which is incorporated herein by reference).

The patches 10 will generally be placed in direct contact with the body. This will be the case for embodiments in which electrical physiological signals are measured, and where the patch incorporates the sensor. In other embodiments, the patch 10 is in proximity of the body, but not in direct contact. For example, there may be no need for direct contact for applications such as the measurement of SpO₂ where the patch 10 may be placed next to a finger-tip assembly consisting of the LED/photodiode combination.

In some embodiments, the patch 10 consists of the ASIC chip 18 in the patch 10, with multiple metal-electrodes (along with electrode-gel) 16 on one side to pick up sensor signals from the body, and PCB trace antenna on the output side of the patch 10 to radiate the radio (RF) signal. A suitable patch of this type of patch is described in U.S. Publication 20140088398, and U.S. Pat. Nos. 8,628,020 and 8,926,509, all of which are incorporated by reference herein in their entirety. Additionally, U.S. Pat. No. 6,215,403 discloses a battery 20 for use in powering the sensor 16 for monitoring blood oxygen content, and temperature, which can also be used in conjunction with the patch 10 of the present invention.

In one embodiment, an alert 22 on the patch 10′ is used to provide confirmation of proper patch placement to the user at the time of patch placement. For example, the patch 10′ may monitor test signals to confirm correct placement. The confirmation of correct placement can be carried out in some cases by the patch 10′, and in some cases with a combination of a base device and the patch 10′, e.g. by holding the base device near the patch. An audio-alert at the patch 10′ could be used for when a patient goes out of the effective range of the base device.

In some embodiments, one or more monitoring devices 16 or mote-based sensors are attached to a patient. The monitoring device 16 senses an electrical signal associated with patient vital sign, locally processes or conditions the signal, and then wirelessly transmits the signal to a central collection point for further processing and/or diagnosis. The sensed signal having patient vital sign data could be processed locally within the monitoring device 16, e.g., by a digital processor or microprocessor, and then 3.0 transferred via a wired connection and/or wirelessly by a transceiver.

Additionally, the patch 10 of the present invention can also provide a completely automated, miniaturized agent delivery system/device 24 capable of detecting, monitoring, and delivering different types of agents from or into a minute amount of fluid. The present invention can determine a subject's reaction to various agents, analyze trends, perform comparisons among a normalized standard of people, determine tolerance levels of a subject, and/or treat the disease or condition accordingly. More specifically, the present invention can include a micro-electro-mechanical system (MEMS) based agent delivery device with optionally integrated fluid acquisition or microfluidic system and external monitoring system. Further, the delivery system can detect a signal and deliver an agent based upon analysis of the chemical concentration. For example, elevated levels of a hormone could result in the delivery of a counteractive medicine, or increased blood pressure could result in the delivery of a blood pressure lowering medication. The delivery system can be controlled externally via the wireless system as disclosed below or can be controlled via a feedback loop contained within the patch itself with reporting data being provided as disclosed herein. Examples of delivery systems are known to those of skill in the art. Some examples of such delivery systems are disclosed in U.S. Pat. No. 5,895,369, U.S. Pat. No. 9,005,527, US Pub. No. 2008/0154179, and US Pub. No. 2009/0143761 all of which are incorporated herein by reference.

The agent delivery device 24 includes an agent delivery reservoir containing the agent to be administered to the patient. An electrolyte receives at least a portion of the agent from the agent delivery reservoir. The electrolyte is mixed with the agent to form an electrolyte-agent mixture that is contained in the reservoir. Moreover, the electrolyte-agent mixture traps the agent until electric current is applied thereto. The device 24 also includes an agent delivery surface in communication with the electrolyte. In a refinement, the agent delivery device 24 includes one or more additional delivery surfaces. The agent delivery surface contacts the patient and delivers agent received from the reservoir to the patent. A controller in communication with the electrolyte-agent mixture provides a series of control pulses to the electrolyte. Each pulse allows the device to administer a portion of the agent to the patient. The series of pulses provides a temporally varying concentration of agent in the patient. In a variation, the electrolyte comprises an iontophoretic electrically conductive material. In a further refinement, the electrolyte is polymeric. The term iontophoretic electrically conductive material means any material that exhibits iontophoretic behavior.

The agent delivery device 24 for use in the patch 10 of the present invention includes a digital controller and a memory accessible to the digital controller. An algorithm for controlling the electrolyte is encoded in the memory such that the algorithm may be executed by the digital controller. The algorithm provides a calculation for the pharmaceutically effective dosing/blood concentration required by the patient. The pharmaceutically acceptable dose or concentration is that dose/concentration that is considered by one of skill in the art to be sufficient to create the appropriate biological and/or chemical response. In a refinement, one or more intervals of the series of controlled pulses are varied over time via the controlling algorithm. In another refinement, the amplitudes of the series of controlled pulses are varied via the controlling algorithm. In still another variation, the duration or width of the series of controlled pulses is varied via the controlling algorithm.

The agent delivery device 24 further includes a sensor system for determining the concentration of the agent in the patient. In a refinement of this variation, such a sensor system is advantageously used in a feedback loop to the controller. In such a feedback loop, information from the sensor system is used to adjust the concentration of the agent or one more additional agents in the patient. In other words, the sensor first detects the concentration of agent in the patient, analyzes this concentration against the predetermined pharmaceutically acceptable dose/concentration, and provides additional agent as required to achieve the pharmaceutically acceptable dose/concentration.

The present invention also provides a method of delivering a biologically compatible agent to a patient utilizing the agent delivery device 24 set forth above. The method of this embodiment includes contacting the patient with the agent delivery device 24 and then utilizing the controller to administer the agent to the patient.

In an alternative embodiment, the agent delivery device 24 can be small and non-invasively monitors interstitial fluids that are in equilibrium with the concentration in blood. This agent delivery device 24 contains a low power micro-fluidic pump for transporting fluid sample to the sensors, micro-fluidic conduits and valves for routing sample and calibration solutions, silver/silver chloride (Ag/AgCl) reference electrodes for electrical stimulation of the skin, microscopic semiconductor sensors to detect ions and chemicals, and electronic circuitry to control the pumps and valves as well as to provide integration with existing data-logging and telemetry systems.

The agent delivery device 24 of the present invention can incorporate microscopic, interdigitated sensor arrays (potentiometric, amperometric, and optical) able to transduce compositions in less than 1 μl sample volumes. Membranes are placed onto the sensing arrays to confer specificity to the desired agent (in combination with other molecules). The agent delivery device 24 is preferably formed utilizing a micro screen printer. Because of their extremely small size, arrays of these sensors provide the ability to utilize more than one electrode for statistical control, as well as providing the ability to transduce dozens of molecules simultaneously.

When iontophoresis has been used to obtain transdermal interstitial fluid samples in the prior art devices, a troublesome tingling sensation was experienced by patients from the large area electrodes employed in the study (10 cm²). Such problems are overcome by the agent delivery device of the present invention, which has a smaller area electrode (1 cm²) with an equivalent current density that does not produce as significant a “side-effect”; however, the reduced surface area results in a significantly reduced volume of drawn interstitial fluid. By reducing the test volume required for analysis by three orders of magnitude, the surface area of the agent delivery device can be significantly reduced without affecting the ability of the agent delivery device to perform the necessary functions. The agent delivery device 24 is able to be so much smaller because of the microscopic semiconductor sensor arrays. The agent delivery device 24 continuously monitors interstitial fluid in near real-time, is a small patch, approximately 10 mm×10 mm, that contains low power micro-fluidic pump for transporting fluid samples, micro-fluidic conduits and valves for routing interstitial fluid samples and calibration solutions, platinum electrodes for electrical stimulation of the skin, microscopic semiconductor sensor arrays to detect glucose, ions, and other analytes, and electronic circuitry to control the pumps and valves as well as to provide integration with existing data-logging, telemetry, and device (pump) control systems.

The method of delivering drugs and metabolites to patients using the agent delivery device 24 of the present invention follows normal physiological concentrations patterns, as opposed to super- or pharmaco-physiological concentrations and patterns, the timing of which is based on systemic factors including receptor dynamics, drug clearance, drug half-life, etc. The delivery timing is based on closed-loop feedback via monitoring of the actual delivered molecule (i.e., lithium or nicotine) or by monitoring of a second indicator molecule (i.e., glucose monitoring for insulin administration). This provides “on-demand” delivery of the agent. Further, the “on-demand” delivery of agents/drugs maintains the body load to the therapeutic level as opposed to the great oscillations present when administered orally or via injection. The invention provides pulsatile delivery of the agent/drug and continuous “ramp-down” capability, controlled automatically. With either form of feedback monitoring, the administration of the agent occurs objectively, without requiring a subjective analysis. This aids in limiting overdosing or creating an addiction to an agent, because the administration is based upon readily ascertainable bodily events that can be tested/analyzed objectively. Since only the necessary amount of agent is being administered, lower amounts of agents can be administered. The end result of the delivery methods are fewer side effects, less drug resistance, less increased tolerance to agents, and increasing the number of individuals that are able to benefit from the agents.

In yet another embodiment of the present invention there is provided a monitoring patch 10 that incorporates diagnostic testing within the patch 10. The diagnostic patch 10 includes a device for acquiring a blood sample and at least one testing element, both of which are incorporated into the patch. The blood sampling element can include blood sample as disclosed herein above, for example iontophoresis can be used to obtain transdermal interstitial fluid samples. These fluid samples are in fluid communication with the diagnostic testing devices. Alternatively, the patch can incorporate a small puncturing unit that punctures the skin and obtains the requisite blood sample. The testing occurs in a manner similar to that describes herein above regarding the administration of pharmaceutical agents. Wherein, instead of having the concentration of an analyte being determined within the patch, the present invention tests for the presence of an analyte in the sample. The testing can be an immunoassay, an enzymatic assay, a biochemical assay, or a chemical assay. The test can be used to detect the present of viruses and bacteria to aid in the early detection of disease. For example, the tests can be used to detect MERSA, clostridium difficile, staphylococcus aureus, and other readily contagious diseases.

The current invention relates to patches 10, integrated circuits (chips) 18, systems and methods for a wireless medical signal processing system for health monitoring which can achieve high wireless link reliability/security, low power dissipation, compactness, low cost and supports a variety of sensors for various physiological parameters. One aspect of the invention is a wireless system for monitoring physiological conditions comprising two or more ASIC chips that are designed to work together to optimize the performance of a wireless monitoring system. One of the ASIC chips is designed to be incorporated into a patch attached to a patient (the patch-ASIC chip), and one of the ASIC chips is incorporated into a mobile or stationary device (the base-ASIC chip). Typically, the base-ASIC chip will be incorporated into a device that will tend to be in the vicinity of the patient. The two or more ASIC chips are designed in order to improve the performance of the system by distributing the different aspects of functionality between the different chip types. Thus the ASIC chips are designed to function in an asymmetric manner in which the base-ASIC will perform more of the processing intensive tasks. In some cases, the base-ASIC will perform all or a majority of functions of a particular type, while the patch-ASIC chip may perform all or a majority of functions of another type. This asymmetric design of the sets of ASIC chips can improve the performance of the physiological monitoring system resulting in better management of power, lower cost, and higher reliability.

In one aspect, the base-ASIC chip is designed to coordinate some of the functions on the patch through the patch-ASIC chip. In many cases, the base-ASIC is incorporated such that it has access to much more power and energy than does the patch-ASIC chip. Thus, the system of the current invention comprises an asymmetric system in which the base-ASIC chip takes on more power and processor intensive functions. This approach can result in lower power dissipation at the patch, which can in turn result in a physiological monitoring system in which the patch can collect and transmit data for days or weeks without recharging or replacing batteries. In addition, the base-ASIC can control the flow of data in a network of patches in order to improve the management of data relating to signals, increasing the amount and quality of physiological information. For example, the base-ASIC chip can supervise and control the functions of the patch-ASIC chip, for example by controlling duty cycle, transmission mode, transmission rate, and transmission timing. The base-ASIC and patch-ASIC chips can be designed such that the coding/decoding functions are asymmetric. For example, the patch-ASIC can be built to carry out Turbo encoding, which is relatively simple, and the base-ASIC can be designed to carry out Turbo-decoding which is more complex, and requires more processing power and therefore uses more energy. Another aspect of asymmetric design of the ASIC chips involves providing a complex antenna scheme such as the use of multiple antennas with smart antenna processing on the base-ASIC, and the use of a single antenna with simple processing on the gate-ASIC. Another aspect of the asymmetric design is the use of different radio scheme capability on the gate-ASIC and base-ASIC chips. For example, low-complexity transmitters (e.g. UWB) and low-complexity receivers (e.g. Narrow-band) are employed on the on patch-ASIC; and high-complexity transmitters (e.g. Narrow band) and high-complexity receivers (e.g. UWB) are employed on the base-ASIC. Another aspect of the asymmetry has to do with distributing the functions of analyzing and controlling the radio channel. Here, the base-ASIC has all the processors for analyzing the radio environment and sending instructions to patch-ASIC to use a particular radio scheme; and the patch-ASIC has simple circuits to just follow the instructions coming from Base ASIC.

In some embodiments two or more of these distributed aspects are coupled together, for example, where the base-ASIC and patch-ASIC are designed to work together such that the base-ASIC has processors for turbo-decoding, multiple antennas and smart antenna processing capability, and high-complexity transmitters (e.g. Narrow band) and high-complexity receivers (e.g. UWB); and the patch-ASIC has processors for Turbo decoding, the capability of receiving signals from a single antenna, and has low-complexity transmitters (e.g. UWB) and low-complexity receivers.

One aspect of the asymmetric distributed processing of the present invention is a patch-ASIC chip and a base-ASIC chip designed to work together in which the area of the base-ASIC chip is higher than that of the patch-ASIC chip. This difference in area results, for example, from the fact that the base-ASIC chip takes on the more processor intensive operations in carrying out the monitoring of a patient's physical condition. In some embodiments, the area of the base-ASIC chip is more than 2 times the area of the patch-ASIC chip. In some embodiments, the area of the base-ASIC chip is more than 4 times the area of the patch-ASIC chip.

In addition to the patch-ASIC chip and the base-ASIC chip, the system further includes a gate-ASIC chip incorporated into a gate device. In some cases the gate-ASIC chip is attached to a μ-Gate, comprising a printed circuit board with an antenna attached to the printed circuit board. In some embodiments, the gate device acts as an intermediary (gateway), for instance, controlling communication between the base-ASIC chip and the patch-ASIC chip. The gate device is useful, for instance in circumstances where the patient wearing the wireless patch may be moved a distance away from the base-ASIC chip for relatively long time periods. In these situations, the gate-device, which will typically be small enough to, for example, be comfortably carried in a pocket, can communicate with the patch-ASIC chip while the patch-ASIC chip is out of communication range of the base-ASIC chip, continuing to monitor and/or control the functions of the patch, and collect and store data sent by the patch, and be able to forward that data to the μ-Base wirelessly. In general, in systems where one or more gate devices are present, patch devices communicate to the μ-Base via the gate device(s). This helps reduce the power consumption of the patch devices as they can transmit at lower power to communicate with the gate device(s) which are in closer proximity than the μ-Base itself.

In some embodiments the gate devices can be incorporated into patches. In these cases, the gate device can perform its gateway function, and can also perform as a patch by being connected (wired) to sensors and receiving physiological signals. For example, one system of the present invention has multiple patches, each patch comprising a patch-ASIC chip; and a gate device comprising a gate-ASIC chip incorporated into a patch on a patient. The gate device can communicate with the multiple patches on the patient, and the gate device can act as an intermediary between the patches comprising the patch-ASICs and a μ-Base comprising a base-ASIC. In one embodiment of this system, the patch-ASICs communicate only by UWB, while the gate-ASIC can communicate with the base-ASIC by either UWB or narrowband. This system allows the patches with patch-ASICs to operate at low power and to have low energy usage by transmitting only in UWB. In this embodiment, the gate-device may have a larger battery than the patches comprising the patch-ASIC chips. In some embodiments, the patch-ASIC chips used in this scenario can be made inexpensively due to the fact that the chips do not have multiple radios. In other embodiments, the patch-ASIC chip and the gate-ASIC chip used in this scenario are made as part of an ASIC superset, where the narrowband radio can be turned off on the patch-ASIC chip. The use of an ASIC superset chip can be advantageous where cost can be driven down by producing a higher volume of a single type of chip.

In other embodiments, the gate device acts mainly as a storage device for physiological information generated and transmitted by the patch. For example, the gate device can have a memory storage capability for storing data transmitted wirelessly from the patch to the gate device. In this embodiment, the gate need not send data wirelessly, and the stored data can be retrieved via a physical connection to another device. For example, the gate device may have a removable memory device, or the gate device may have a connector which allows it to be connected to another device in order to download the information on the gate device to another device, such as a medical device or computer.

Additionally, the patch can include communication modules that enable communication between the health care professional and the patch user. Examples of such modules are well known to those of skill in the art and can include, but are not limited to, a small speaker system, such as piezoelectric speaker panels and microphones contained within a micro-chip that is located within the patch.

The data obtained from the patch can be transported or “shared” via secure communications means that is HIPAA compliant. One example of such a secure communication system is provided by Ultralinq, as disclosed on the website (www.ultralinq.com). Other forms of secure communication means are well known to those of skill in the art and include, but are not limited to, via cloud communications, via wireless chips, and other similar secure wireless systems.

The transported data can then be processed using assistive interpretation. Examples of assistive interpretation are disclosed in U.S. Pat. No. 9,135,399 and U.S. Pat. No. 9,361,430. In other words, the data can be used to help differentiate between conditions or diseases. Also, the data can be used to help differentiate between healthy and non-healthy states. The assistive interpretation relates to a reconfigurable medical decision support system for processing medical data of a patient. In one embodiment, the system includes a data processing system with a memory in communication with a processor, the memory including program instructions for execution by the processor to receive the medical data; access a knowledge-base data set stored therein, the knowledge-base including a feature set relating to a pathophysiological condition, the feature set having a plurality of associated features, each feature having a plurality of validated quantifiable stages and each validated quantifiable stage being assigned a score, associate the medical data with features of the feature-set, detect a request to modify a value of a validated quantifiable stage associated with a feature, verify the request and modify the validated quantifiable stage to create a modified validated quantifiable stage, associate the score from the validated quantifiable stage with the modified validated quantifiable stage, determine a medical risk value based on the modified validated quantifiable stage and the assigned score, determine a medical finding from the knowledge base corresponding to the medical risk value, associate an output statement stored in the knowledge-base with the medical finding, and a user interface for providing the output statement.

Another aspect of the disclosed embodiments relates to a computer program product. In one embodiment, the computer program product includes computer readable program code means for evaluating medical data of a person to determine a medical finding, the computer readable program code means when executed in a processor device, being configured to, obtain the medical data of the person, access a medical knowledge-base data set stored in a memory, the knowledge-base including a feature set relating to a pathophysiological condition, the feature set having a plurality of associated features, each feature having a plurality of validated quantifiable stages and each validated quantifiable stage being assigned a score, enable a user to reconfigure a value associated with a validated quantifiable stage associated with a feature, associate the medical data with features of a feature-set from the knowledge-base, determine an association between the medical data and the quantifiable stages associated with the feature corresponding to the medical data, determine scores corresponding to the association of the medical data and quantifiable stages, determine a medical risk value based on the scores corresponding to the association of the medical data and quantifiable stages, determine a medical finding from the knowledge-base corresponding to the medical risk value, and provide an output statement corresponding to the medical finding.

An exemplary configurable or reconfigurable diagnostic decision support and medical finding prediction system incorporating aspects of the present disclosure is shown. In operation, the diagnostic decision support and medical finding prediction system of the disclosed embodiments generally comprises a dynamically layered clinical/computer decision support system (CDSS) that provides automated decision support to assist healthcare providers in the screening, evaluation and diagnoses of medical or disease conditions. The diagnostic decision support and medical finding prediction system of the disclosed embodiments is configurable, or reconfigurable, and will generally be referred to herein as a “configurable medical prediction system.” The aspects of the disclosed embodiments allow users to reconfigure key inputs and outputs of the configurable medical finding prediction system's knowledge based algorithms. This function allows users of a pre-determined algorithm to directly influence the data ranges and output expressions without adversely affecting the power of the decision making process. The aspects of the disclosed embodiments assure the user's dominant role in the decision making processes of the configurable medical finding prediction system.

In one embodiment, the exemplary configurable medical finding prediction system includes a medical data acquisition system or tool and a data processing system. Although the data acquisition tool can be a stand-alone device, in one embodiment, the data acquisition tool can also be integrated into the data processing system.

The data acquisition system is configured to obtain or acquire medical and diagnostic data of a person, also referred to herein as a “patient.” The medical and diagnostic data, generally referred to as “medical data”, can generally include any patient examination results or data, and diagnostic information and parametric, which reflect a physiological state or condition of the patient. Examples of the medical data can include for example, but are not limited to, vital sign data, electrocardiogram (ECG) data, laboratory and examination results, diagnostic test data and diagnostic imaging data, etc. In alternate embodiments, the medical data can include any health or diagnostic data related to or otherwise associated with the patient.

The source of the medical data that is obtained by the data acquisition too′ and/or provided to the data processing system can include any suitable diagnostic device or system that is configured to obtain physiological and other medical related information and data of a patient. Examples of these types of devices and systems can include, but are not limited to, clocks, timers, blood pressure monitors, electrocardiogram (ECG) monitors, echocardiogram and Doppler devices, ultrasound systems, magnetic resonance (MR) systems, computer tomography (CT) systems, positron emission tomography (PET) systems, ventilation monitors, blood analysis devices, drug and fluid dispensing devices, blood sugar monitors, temperature monitors, telemetry units, pulse oximetry devices, diagnostic imaging devices, electronic medical records, plans of care, disease templates and protocols, etc. In alternate embodiments, the source of the medical data obtained by the data acquisition tool can include any suitable source of medical data and health related information. The data acquisition tool is configured to obtain the medical data in any suitable or known fashion. In one embodiment, the data acquisition tool includes or is communicatively coupled to one or more of the sources of the medical data. For example, the data acquisition tool can receive diagnostic data directly from a diagnostic device such as a sonogram or x-ray system, in the form of a data transfer or download. Alternatively, the data acquisition tool can access or obtain the medical data from a memory storage device or system that is used to store the medical data, such as a health information processing and storage system or device or electronic medical record. In one embodiment, the medical data can also be manually inputted by the clinician to the data acquisition tool. The aspects of the disclosed embodiments are not intended to be limited by the manner in which the data acquisition tool obtains the medical data and other health related information for processing in the system. In one embodiment, the data acquisition tool is part of a hospital data or medical record network or such other suitable medical record and information network, and is configured to receive and transmit data and information, as well as store such information. The data acquisition tool can also include one or more processors comprised of or including machine-readable instructions that are executable by a processing device. Alternatively, the data acquisition tool can incorporate algorithms that can be used to predict or diagnose the presence of a disease or -pre-disease state (examples of which include, but are not limited to, sepsis, systemic inflammatory response syndrome, graft versus host disease, jaundice, and other monitorable conditions). For example, the algorithm could be a known algorithm for early prediction of sepsis in a patient recently released from the hospital. Such algorithms are known to those of skill in the art.

The data acquisition tool is communicatively coupled to the data processing system. The data processing system is configured to receive and process the medical data and provide outputs, or expressions of outputs that can be used by the clinician in evaluating the medical data and generating a diagnosis. The data processing system generally comprises a memory, a processor and a user interface. The user interface includes or is coupled to an output or display device. The display device can also comprise an audio component. In one embodiment, the user interface and display device comprises a single interface between the data processing system and the user or clinician. Although the memory, processor and user interface are shown as being part of the same device, in alternate embodiment each could be part of a separate device or system.

In use, the system functions as follows. A patch is provided to a patient. This can occur in a hospital, at an outpatient facility or via the mail. The patient then applies/attaches or has attached the patch at the appropriate location for obtaining the necessary medical information. The application of the patch is described in materials provided with the patch to the patient. Once the patch is properly affixed, the patient will be provided with an indicator that confirms proper application of the patch. The indicator can be any indicator known to those of skill in the art and can include, but is not limited to, a light, a color change, a sound, and message that is received by the patient or health care provider, or any other similar indicator.

Once the patient received the indication that the patch is properly affixed, the system can begin to transmit the medical information. This can either be an automatic transmission that commenced after the indication is given to the patient or there can be a “start” signal that is provided by the patient to commence the transmission of the information. The “start” signal can be provided from a communication from the patient and can be a button, a lever, the closing of a circuit that is only effectuated once the patch is properly affixed, or another signal that is transmitted from the patient to indicate that the communication should begin.

Once the communication begins, the medical information starts being transmitted it will continue to be transmitted through the secure system until either the patch is removed or turned off. The data can be transmitted continuously, sporadically, at predetermined intervals, or as requested by the doctor or patient.

The transmitted information is then processed using the assistive interpretation module, which information can be transmitted to the doctor or other health care professional. The health care professional can the provide patient feedback either through the secure communication system, via a feedback loop within the system as described in detail herein, or via another other known communication systems. Alternatively, the patient can receive communications from the assistive interpretation module to indicate the patient's status. For example, the patient can receive a signal that indicates all is well, that the patient should call a health care provider, or seek immediate medical attention. The system can also include a safety module that contacts a local emergency medical unit to indicate that the patient is in need of immediate medical attention.

Throughout this application, author and year and patents by number reference various publications, including United States patents. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used herein, is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

What is claimed is:

1. A wireless patch comprising:
a patch body;
an adhesive material located on a bottom surface of said patch body;
at least one sensor contained within said patch body;
wireless data transfer means for transferring data from said sensor, said wireless data transfer means located within said patch body to an outside source.

2. The patch according to claim 1, wherein said sensor is a sensor selected from the group consisting essentially of potentiometric sensors, amperometric sensors, optical sensors, biochemical sensors, temperature sensors, electrical sensors, chemical sensors, and combinations thereof.

3. The patch according to claim 1, wherein said wireless data transfer means is an application-specific integrated chip.

4. The patch according to claim 1, further including a feedback loop for interpreting the data received from said sensor and effectuating a response to the interpretation.

5. The patch according to claim 4, wherein said feedback loop includes a reservoir within said patch body in communication with said sensor, wherein when a signal is provided said reservoir releases an agent through said adhesive layer.

6. A wireless medical system for remotely monitoring, detecting, and/or diagnosing disease in a patient, the system comprising:
at least one patch for sensing and communicating data;
data transfer means for transferring the data from said patch;
data receiving means for receiving the data from said patch and monitoring, detecting, and/or diagnosing disease in a patient.

7. The wireless system according to claim 6, wherein said patch is the patch according to claim 1.

8. The wireless system according to claim 7, wherein said patch includes signal means for insuring proper placement of said patch on the patient.

9. The wireless system according to claim 8, wherein said signal is selected from the group consisting essentially of an audio signal, a visual signal, and combinations thereof.

10. The wireless system according to claim 6, including at least two patches located at distinct locations on the patient.

11. The wireless system according to claim 6, wherein said data transfer means is an application-specific integrated chip.

12. The wireless system according to claim 6, wherein said data receiving means includes assistive interpretation software for receiving the data from the patch for detecting and diagnosing disease in the patient and creating a resultant diagnosis.

13. The wireless system according to claim 12, wherein said data receiving means further includes communication means for transmitting the diagnosis of the patient.

14. A method of monitoring, detecting, and/or diagnosing disease in a patient by:
affixing at least one patch according to claim 1 to the patient;
monitoring the signals being sensed by the patch;
transmitting the signals to a data receiver;
receiving the signals from the patch, interpreting the signals, and generating a resulting diagnosis; and
transmitting the diagnosis of the patient.

15. The method according to claim 14, wherein said interpreting step includes inputting the transmitted signals into assistive interpretation software for generating the resulting diagnosis.

16. The method according to claim 14, wherein said affixing step include placing the patch on the patient and generating a confirmatory signal indicating proper placement of the patch on the patient.

17. The method according to claim 15, further including a feedback signal for generating a localized treatment based on the diagnosis.

18. The method according to claim 16, wherein said affixing step includes affixing two patches to the patient for monitoring separate locations on a patient.
